(19) United States
(12) Patent Application Publication
(10) Pub. No.: US 2003/0133995 A1
(43) Pub. Date: Jul. 17, 2003

(54) Ingestible capsaicin neutralizer

(76) Inventors: David K. Mellott, San Antonio, TX (US); Aren J. Mellott, San Antonio, TX (US)

Correspondence Address: GUNN, LEE & HANOR, 700 N. ST. MARY'S STREET, SUITE 1500, SAN ANTONIO, TX, 78205, US

(21) Appl. No.: 10/051,670

(22) Filed: Jan. 17, 2002

(51) Int. Cl. A61K 38/16; A61K 33/14; A61K 31/70

(52) U.S. Cl. 424/679; 424/680; 514/23; 514/21

(57) ABSTRACT

A chemical composition for an ingestible capsaicin neutralizer to neutralize the effect of capsaicin on the oral cavity, tongue, and esophagus when capsaicin from hot peppers is ingested by a user comprised of an effective neutralizing amount of casein protein, or the salt thereof, an alkali earth metal halide, and the balance water.

### BACKGROUND OF THE INVENTION

1. Field of the Invention

Applicant's invention relates to an ingestible therapeutic composition to neutralize the effect of capsaicin from hot peppers on the oral cavity, tongue, and esophagus when capsaicin is ingested by a user. This composition comprises an effective neutralizing amount of casein protein, or the salt thereof, an effective amount of alkali earth metal halide, and the balance water.

2. Background Information

Many people enjoy eating hot peppers. But unfortunately all hot peppers contain a certain concentration of capsaicinoids, which are the natural substances within the hot peppers that cause the burning sensation within the mouth, causing the eyes to water and the nose to run. This sensitivity to capsaicinoids is due to the human's neural receptors that respond to warm stimuli which have a peak sensitivity above normal body temperature. These receptors can be found inside the mouth and when exposed to the heat stimuli from the hot peppers will respond to the heat stimuli as long as they are in contact with the hot pepper. The heat level of these hot peppers is measured in Scoville units and can range from 100 for Bell peppers to 300,000 for Habanero peppers.

There are several types of capsaicinoids that exist in hot peppers. The minor capsaicinoids that exist include nordihydrocapsaicin, homocapsaicin, and homodihydrocapsaicin and are illustrated by their chemical structures below.

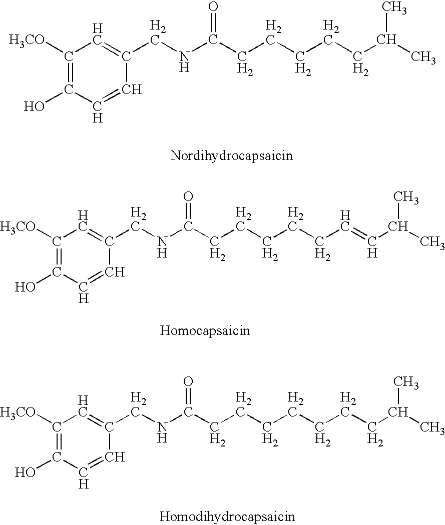

The most common and the most pungent capsaicinoid is capsaicin, also known as N-Vanillyl-8-methyl-6-(E)-noneamide. Capsaicin is synthesized in the placenta of the pepper on which the seeds grow. The capsaicin then slowly permeates through the remainder of the pepper depositing more or less of the compound in the remaining parts. The heat sensation of pure capsaicin is approximately 16,000,000 Scoville units and is so hot that in its pure form diluted one hundred thousand fold it can cause blistering of the tongue. Capsaicin and the compound dihydrocapsaicin together make up about 80-90% of the capsaicinoids found in peppers and can be present in a 1:1 or 2:1 ratio. The capsaicinoid content in many varieties of hot peppers can range from 0.1 to 1.0%.

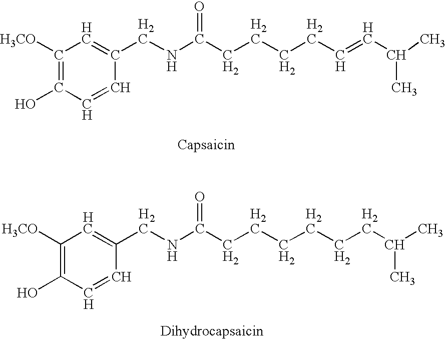

In order for a typical individual to enjoy the taste of hot peppers with such a concentration of capsaicin it becomes prudent for a composition to exist that can quickly neutralize the capsaicin heat on the oral cavity, tongue, and esophagus. No such product exists on the market currently. The present composition was created to satisfy this long felt need in the marketplace.

### SUMMARY OF THE INVENTION

The present invention is an ingestible therapeutic composition to neutralize the effect of capsaicin on the oral cavity, tongue, and esophagus when capsaicin from hot peppers is ingested by a user. This composition comprises an effective neutralizing amount of casein protein, or the salt or mixture of salts thereof, an effective amount of alkali earth metal halide, and the balance water.

More particularly, the present invention is an ingestible therapeutic composition to neutralize the effect of capsaicin on the oral cavity, tongue, and esophagus when capsaicin from hot peppers is ingested by a user, having an effective neutralizing amount of calcium, sodium, or potassium caseinate, or a mixture thereof, an effective amount of sodium or potassium chloride, and the balance water.

In addition, the present invention is an ingestible therapeutic composition to neutralize the effect of capsaicin on the oral cavity, tongue, and esophagus when capsaicin from hot peppers is ingested by a user, having an effective neutralizing amount of calcium, sodium, or potassium caseinate, or a mixture thereof, an effective amount of sodium or potassium chloride, and the balance an aqueous acid such as citrus extract.

Next, the present invention is a method for preparing an ingestible therapeutic composition to neutralize the effect of capsaicin on the oral cavity, tongue, and esophagus when capsaicin from hot peppers is ingested by a user. This method includes mixing together an effective neutralizing amount of casein protein, or the salt or mixture of salts thereof, and an effective amount of alkali earth metal halide in a water base. A pharmaceutically acceptable carrier, diluent, or excipient may be added.

Further, the present invention relates to a method for preparing an ingestible therapeutic composition to neutralize the effect of capsaicin on the oral cavity, tongue, and esophagus when capsaicin from hot peppers is ingested by a user. The method includes mixing an effective neutralizing amount of calcium, sodium, or potassium caseinate, or a mixture thereof, and an effective amount of potassium or sodium chloride in a water base. A pharmaceutically acceptable carrier, diluent, or excipient may be added.

In addition, the present invention is a method for preparing an ingestible therapeutic composition to neutralize the effect of capsaicin on the oral cavity, tongue, and esophagus when capsaicin from hot peppers is ingested by a user. The method includes mixing an effective neutralizing amount of calcium, sodium, or potassium caseinate, or a mixture thereof, and an effective amount of sodium or potassium chloride in a base of aqueous acid such as citrus extract. A pharmaceutically acceptable carrier, excipient, or diluent may be added.

Further, the present invention relates to a method for dispensing an ingestible therapeutic composition to neutralize the effect of capsaicin on the oral cavity, tongue, and esophagus when capsaicin from hot peppers is ingested by a user. This method may include dispensing the ingestible therapeutic composition in a solid or liquid form. The composition so dispensed having an effective neutralizing amount of calcium, sodium, or potassium caseinate, or a mixture thereof, an effective amount of sodium or potassium chloride, and the balance water. Such a composition may include a pharmaceutically acceptable carrier, diluent, or excipient.

In addition, the present invention relates to a method for dispensing an ingestible therapeutic composition to neutralize the effect of capsaicin on the oral cavity, tongue, and esophagus when capsaicin from hot peppers is ingested by a user. This method may include dispensing the ingestible therapeutic composition in a solid or liquid form. The composition so dispensed having an effective neutralizing amount of calcium, sodium, or potassium caseinate, or a mixture thereof, an effective amount of sodium or potassium chloride, and the balance an aqueous acid such as citrus extract. Such a composition may include a pharmaceutically acceptable carrier, diluent, or excipient.

### DETAILED DESCRIPTION OF THE PREFERRED EMBODIMENT

The present invention is an ingestible therapeutic composition for neutralizing the effect of capsaicin on the oral cavity, tongue, and esophagus when capsaicin from hot peppers is ingested by a user as well as a method for preparing and dispensing the composition. It is believed that the present invention has a similar neutralizing effect on capsaicin in the stomach. Studies are currently underway to examine this hypothesis. Capsaicin in its pure form is not very soluble in aqueous solutions, but is quite soluble in fats, oils and alcohols. Capsaicin solubility and rate of exchange can vary when it is present in the hot pepper. When the capsaicin leaches from a hot pepper the rate of transition from the pepper to the user's oral cavity, tongue, esophagus, and stomach can be so slow in some varieties as to allow for some temporary dissolution and/or neutralization in an aqueous solution until the concentration reaches some predetermined threshold.

The preferred composition of the present invention comprises an effective neutralizing amount of casein protein, or the salt or mixture of salts thereof. This effective neutralizing amount is about 0.1 to 100% by weight of the present invention. Casein protein is the principal protein in milk. It acts as a storage protein being the major nitrogen source for mammalian infants. The effective neutralizing amount of casein protein in the present therapeutic composition can also be in the form of a salt to render it more soluble. The salt form of casein protein is in the form of an alkali or alkaline earth metal caseinate, preferably sodium, potassium or calcium caseinate, or a mixture thereof.

Further, the therapeutic composition contains an effective amount of alkali earth metal halide to control the capsaicin. The preferred alkali earth metal halides are sodium and potassium chloride; the most preferred being sodium chloride.

The balance of the therapeutic composition is water in an amount sufficient to give the final therapeutic composition the consistency of a solid (0% added water), paste, gel, suspension or solution. An aqueous acid such as from a citrus extract, preferably lime or lemon extract, can be substituted for the water where applicable. The ultimate consistency will dictate the method by which the final therapeutic composition is dispensed. It is presumed that when the first two ingredients are present in the solid form, which may include a powder formulation, the balance of water may come from the user's saliva.

In preparing the final ingestible therapeutic composition to neutralize the effect of capsaicin on the oral cavity, tongue, and esophagus when capsaicin from hot peppers is ingested by a user, an effective neutralizing amount of calcium, sodium, or potassium caseinate, or a mixture thereof being about 0.1 to 100% by weight in the composition is mixed with the remainder to 100%, if any, of an effective amount of potassium or sodium chloride in a water base.

While it is preferred that the final therapeutic composition contain only the above listed ingredients, it will be understood by those skilled in the art that in order to form a therapeutic agent the final therapeutic composition may be present in an amount of from about 0.1 to 98% by weight with the balance being a pharmaceutically acceptable carrier, diluent, or excipient. Solid formulations of the composition for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that may be used include, without limitation, microcrystalline cellulose, corn starch, sodium starch, glycolate, and alginic acid. Tablet binders that may be used include acacia, methylcellulose, sodium carboxymethylcellulose, polyvinylpyrrolidone (Povidone™), hydroxypropyl methylcellulose, sucrose, starch, and ethylcellulose.

Liquid formulations of the composition for oral administration prepared in water or other aqueous vehicles such as the aqueous acid from the citrus extract may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups, and elixirs containing, together with the active compound(s), wetting agents, sweeteners, and coloring and flavoring agents.

A lip balm can also be prepared from the final therapeutic composition in a suitable cosmetic medium, which does not react with the final therapeutic composition and is otherwise toxicologically and pharmaceutically acceptable. Preferred examples include petrolatum; modified or unmodified vegetable oils such as peanut oil, wheatgerm oil, linseed oil, jojoba oil, apricot kernel oil, walnut oil, palm oil, pistachio oil, sesame oil, cade oil, grapeseed oil, avocado oil, soy oil, sweet almond oil, calophyllum oil, castor oil, olive oil, sunflower oil; animal oils; synthetic silicon oil such as dimethylpolysiloxane; alkyl and alkenyl esters of fatty acids such as isopropyl esters of myristic, palmitic and stearic acids and fatter esters which are solid at room temperature; waxes such as lanolin wax, candelilla wax, cocoa butter, karite butter, silicon waxes, hydrogenated oils which are solid at room temperature, sucroglycerides, oleates, myristates, linoleates, stearates, paraffin, beeswax, carnauba wax, ozokerite, microcrystalline wax; fatty alcohols such as lauryl, cetyl, myristyl, stearyl, palmityl, and oleyl alcohols; polyoxyethylated fatty alcohols; and wax esters, lanolin and its derivatives, perhydrosqualene and saturated esters, ethyl palmitate, isopropyl palmitate, alkyl myristates such as isopropyl myristate, butyl myristate, and decyl myristate, hexyl stearate, triglyceride esters, triglycerides of octanoic and decanoic acid, cetyl ricinoleate, stearyl octanoate, fatty acids, polyhydric alcohols, polyether derivatives, fatty acid monoglycerides, polyethylene glycol, propylene glycol, alkyl ethoxy ether sulfonates, ammonium alkyl sulfates, fatty acid soaps, and hydrogenated polyisobutene and mixtures of waxes and oils. The lip balm can also contain various ingredients usually contained in cosmetic and lip balm compositions such as dyestuffs, pigments, perfumes, and preservatives. Such dyestuffs or pigments may be dissolved or suspended in the medium and can be insoluble lakes or oil soluble dyes of the eosin group, or both. It may also be possible to include additives which increase protection against sunburn, such as UV absorbers, coffee oil, derivatives of salicylic acid, derivatives of cinnamic acid, derivatives of para-aminobenzoic acid (PABA), derivatives of benzophenone and derivatives of camphor. Further optional ingredients may include chelating agents such as gluconic, citric and tartaric acids, thickening agents such as cross-linked carboxyl polymethylene polymers, bentonite and gums, emulsifiers, fragrance materials, vitamins, aloe vera, water, alcohol or acetone as necessary to achieve the desired results.

A carbohydrate or carbohydrate alcohol may be added to the final therapeutic composition or therapeutic agent to soften or sweeten the taste of the casein protein, or the caseinate salt or mixture of caseinate salts thereof. While the carbohydrate or carbohydrate alcohol is not necessary for the therapeutic properties of the composition, it is preferred to improve the taste of the composition. A limited amount of carbohydrates, specifically sugars, exist that are used extensively as sweeteners and can be used in the present composition. These include sucrose, glucose, invert sugar (glucose/fructose mixture), maltose, lactose, and fructose. In addition, sugars and sugar alcohols that are used in dietary products could be used in the present composition as well. These include sorbitol, xylitol, mannitol, maltulose, isomaltulose, maltitol, isomaltitol, lactulose, and lactitol.

Since the therapeutic effect of the final therapeutic composition begins in the oral cavity, it is not anticipated that the present invention will be developed into an injectable formulation. Regardless of the final form of the composition for administration, the final therapeutic composition or therapeutic agent will be administered on an as needed basis by the user. The amount necessary for relief will vary based on the body weight of the user as well as the user's developed capsaicin tolerance.

Tests were performed with the composition of the present invention to determine its effectiveness as an ingestible therapeutic composition to neutralize the effect of capsaicin on the oral cavity, tongue, and esophagus. Surprisingly, in one test, Subject 1 chewed and swallowed pieces of a Serrano pepper (Scoville units=15,000) which is hotter than a jalapeno pepper. Once the heat sensation had developed to its maximum, Subject 1 sipped the composition in liquid form, rinsed the composition around in his mouth and swallowed the composition. This procedure was repeated until the heat sensation dampened. Within one minute Subject 1 no longer felt the extreme heat sensation effect of the Serrano pepper. Within five minutes, all heat sensation from the Serrano pepper had disappeared. Additional tests performed with jalapeno peppers (Scoville units=5,000) yielded similar results.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A chemical composition for neutralizing the effect of capsaicin from hot peppers on the oral cavity, tongue, and esophagus when capsaicin is ingested by a user comprising an effective neutralizing amount of casein protein, or the soluble salt thereof, in admixture with an effective amount of alkali earth metal halide.

2. The chemical composition for neutralizing the effect of capsaicin from hot peppers on the oral cavity, tongue, and esophagus when capsaicin is ingested by a user of claim 1 wherein said admixture of casein protein, or the soluble salt thereof, and alkali metal halide are combined in a water base.

3. The chemical composition for neutralizing the effect of capsaicin from hot peppers on the oral cavity, tongue, and esophagus when capsaicin is ingested by a user of claim 2 further comprising an effective amount of sugar or sugar alcohol as a flavoring additive.

4. The chemical composition for neutralizing the effect of capsaicin from hot peppers on the oral cavity, tongue, and esophagus when capsaicin is ingested by a user of claim 3 wherein said alkali earth metal halide is sodium chloride or potassium chloride.

5. The chemical composition for neutralizing the effect of capsaicin from hot peppers on the oral cavity, tongue, and esophagus when capsaicin is ingested by a user of claim 4 wherein said soluble salt thereof is selected from the group consisting of sodium caseinate, potassium caseinate, and calcium caseinate, or a mixture thereof.

6. The chemical composition for neutralizing the effect of capsaicin from hot peppers on the oral cavity, tongue, and esophagus when capsaicin is ingested by a user of claim 5 wherein said sugar is selected from the group consisting of sucrose, fructose, and glucose.

7. The chemical composition for neutralizing the effect of capsaicin from hot peppers on the oral cavity, tongue, and esophagus when capsaicin is ingested by a user of claim 5 wherein said sugar alcohol is selected from the group consisting of sorbitol, xylitol, and mannitol.

8. A pharmaceutical preparation for neutralizing the effect of capsaicin from hot peppers on the oral cavity, tongue, and esophagus when capsaicin is ingested by a user comprising a compound according to claim 1, in admixture with a pharmaceutically acceptable carrier, diluent, or excipient.

9. The pharmaceutical preparation for neutralizing the effect of capsaicin from hot peppers on the oral cavity, tongue, and esophagus when capsaicin is ingested by a user of claim 8 wherein said soluble salt thereof is selected from the group consisting of sodium caseinate, potassium caseinate, and calcium caseinate, or a mixture thereof.

10. The pharmaceutical preparation for neutralizing the effect of capsaicin from hot peppers on the oral cavity, tongue, and esophagus when capsaicin is ingested by a user of claim 9 further comprising an effective amount of sugar or sugar alcohol as a flavoring additive.

11. The pharmaceutical preparation for neutralizing the effect of capsaicin from hot peppers on the oral cavity, tongue, and esophagus when capsaicin is ingested by a user of claim 10 wherein said sugar is selected from the group consisting of sucrose, fructose, and glucose.

12. The pharmaceutical preparation for neutralizing the effect of capsaicin from hot peppers on the oral cavity, tongue, and esophagus when capsaicin is ingested by a user of claim 10 wherein said sugar alcohol is selected from the group consisting of sorbitol, xylitol, and mannitol.

13. A method for preparing a chemical composition for neutralizing the effect of capsaicin from hot peppers on the oral cavity, tongue, and esophagus when capsaicin is ingested by a user comprising the steps of mixing an effective neutralizing amount of casein, or the soluble salt thereof, with an effective amount of alkali earth metal halide to form a mixture.

14. The method for preparing a chemical composition for neutralizing the effect of capsaicin from hot peppers on the oral cavity, tongue, and esophagus when capsaicin is ingested by a user of claim 13 further comprising the step of combining said mixture in a water base.

15. The method for preparing a chemical composition for neutralizing the effect of capsaicin from hot peppers on the oral cavity, tongue, and esophagus when capsaicin is ingested by a user of claim 14 further comprising the step of adding an effective amount of sugar or sugar alcohol to said mixture.

16. The method for preparing a chemical composition for neutralizing the effect of capsaicin from hot peppers on the oral cavity, tongue, and esophagus when capsaicin is ingested by a user of claim 15 wherein said alkali earth metal halide is sodium chloride or potassium chloride.

17. The method for preparing a chemical composition for neutralizing the effect of capsaicin from hot peppers on the oral cavity, tongue, and esophagus when capsaicin is ingested by a user of claim 16 wherein said soluble salt thereof is selected from the group consisting of sodium caseinate, potassium caseinate, and calcium caseinate, or a mixture thereof.

18. The method for preparing a chemical composition for neutralizing the effect of capsaicin from hot peppers on the oral cavity, tongue, and esophagus when capsaicin is ingested by a user of claim 17 wherein said sugar is selected from the group consisting of sucrose, fructose, and glucose.

19. The method for preparing a chemical composition for neutralizing the effect of capsaicin from hot peppers on the oral cavity, tongue, and esophagus when capsaicin is ingested by a user of claim 17 wherein said sugar alcohol is selected from the group consisting of sorbitol, xylitol, and mannitol.

20. The method for preparing a chemical composition for neutralizing the effect of capsaicin from hot peppers on the oral cavity, tongue, and esophagus when capsaicin is ingested by a user of claim 17 further comprising the step of adding a pharmaceutically acceptable carrier, diluent, or excipient to said mixture.
